(19) United States
(12) Patent Application Publication
(10) Pub. No.: US 2016/0207059 A1
(43) Pub. Date: Jul. 21, 2016

(54) DEVICE AND METHOD FOR APPLYING FLUIDS IN DIFFICULT ACCESS LOCATIONS

(71) Applicant: SANOFI-AVENTIS DE MEXICO, C.A DE C.V, COYOACAN (MX)

(72) Inventor: PABLO ALVARADO CRUZ, COYOACAN (MX)

(21) Appl. No.: 14/997,148

(22) Filed: Jan. 15, 2016

(30) Foreign Application Priority Data
Jan. 16, 2015 (MX) MX/A/2015/000767

(51) Int. Cl. B05B 11/00; A01N 37/06; A01N 31/16; A61L 2/22

(52) U.S. Cl. CPC B05B 11/3035; A61L 2/22; A01N 37/06; A01N 31/16

(57) ABSTRACT

The present invention relates to the field of devices for applying fluid in difficult access locations. More particularly, the invention reveals a container with a retractable, flexible neck that allows easy application of powder in difficult access locations, such as the posterior region of shoes, as well as a method for applying fluids through the use of this device.

### RELATED APPLICATIONS

This application claims priority to Mexican Patent Application No. MX/a/2015/000767, filed Jan. 16, 2015, which is incorporated herein by reference in its entirety.

### FIELD OF THE INVENTION

The present invention relates to the field of devices for applying fluid in difficult access locations. More particularly, the invention discloses a plastic container with a retractable, flexible neck, providing access to smaller compartments, as the inner parts of footwear, as well as a method for applying fluids through the use of this device, such a container may contain various materials fluently.

### BACKGROUND OF THE INVENTION

The use of deodorant foot powder in footwear is being quite applicable in the prior art. It is a way to prevent the growth of bacteria that cause bad smell, fungal infections and other problems that may develop with the suppression of the foot within the footwear for a long period of time.

There is a difficulty in applying deodorant powder inside shoes, wherein by virtue of the form of footwear, access to the innermost region is damaged, and the product ends up being not applied uniformly throughout its length.

The present invention describes a solution to this problem. The appropriate format for applying the fluid material in footwear is via flexible neck vial, so as to allow the product peak shedding to distribute the material in the innermost region of the shoe.

The prior art has examples of products where the contents are spilled by an extendable and retractable lead. The U.S. Pat. No. 5,097,994 describes a container where the shedding peak includes a medially positioned accordion, an extensible and retractable lead to spill a liquid, as well as a selective orientation of the liquid outpouring.

U.S. Pat. No. 4,492,324 refers to a container suitable for liquids containing a flexible neck in the form of an accordion, attached to the container, however, with a smaller cross area, which can be bent and kept in this position when at rest. This container is able to access difficult access holes, such as automobile engines. US Patent 2004/245208 application describes a bottle used for spraying liquids, comprising a spray jar attached to a flexible tube that allows its movement through the vial centerline in any direction over 360 degrees, with continuous liquid release. The vial is based on dispensing the liquid sprayed in any direction while maintaining the liquid in contact with the sprayer lead, so that the container remains in the vertical position and the sprayer is targeted to any direction.

Considering all the documents submitted, none of them relates to a useful container for applying powder pharmaceutical compositions. The examples presented by documents consist in applying liquid products in automotive engines. Thus, the solution proposed here, in the eyes of the inventors, has all the requirements for patentability before the prior art.

### SUMMARY OF THE INVENTION

In a first aspect, the present invention provides a container for applying fluid in difficult access locations. In a preferred embodiment, the container according to FIG. 2 comprises the retractable and flexible neck (2), ergonomically able to properly fit the user's hand.

The use of the device is an additional object of the present invention, according to FIG. 3, preferably comprising the body (1) where pressure is applied to propel the powder composition within the retractable, flexible neck (2) and the dispenser (3) with the cover (4) for applying the powder composition in the treatment of odors and athlete's foot.

It is an additional object of the present invention and a method for applying fluids in difficult access locations, comprising the steps of:

a) placing the device in the location where the fluid is applied by adjusting the retractable, flexible neck; and

b) applying the fluid to the desired location.

In a preferred embodiment, the applying step is performed by the pressure exerted by the user on the fluid container, thereby expelling the contents.

These and other objects of the invention are immediately assessed by those skilled in the art and by the companies with interests in the segment, and will described in a detailed way, sufficient for its reproduction in the description below.

### DESCRIPTION OF THE DRAWINGS

FIG. 1 shows the body (1) where the pressure for propelling the powder composition is applied therein, with a retractable and flexible neck (2), comprising the dispenser (3) and the cover (4).

FIG. 2 shows a perspective view.

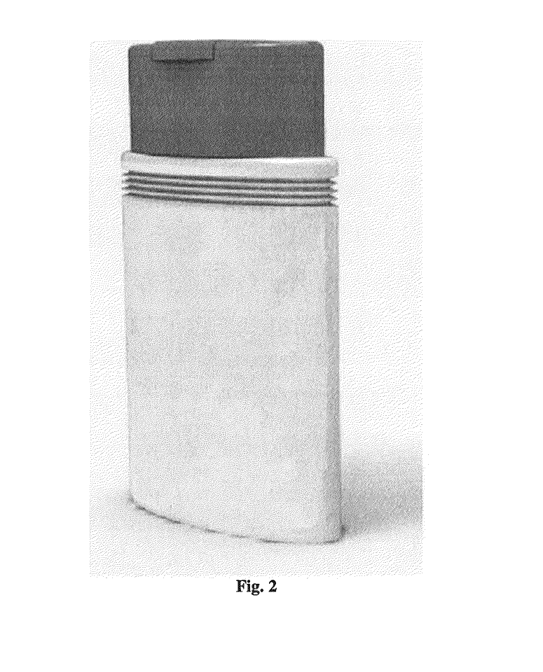

FIG. 3 shows a preferred embodiment for the use of the device in the application of fluids in difficult access locations. By way of example, without limitation, FIG. 3 depicts a shoe in partial cutaway with a device embodying features of the present invention.

FIG. 4 shows a front view (a) and a side view (b) of an expected variation to the neck (2).

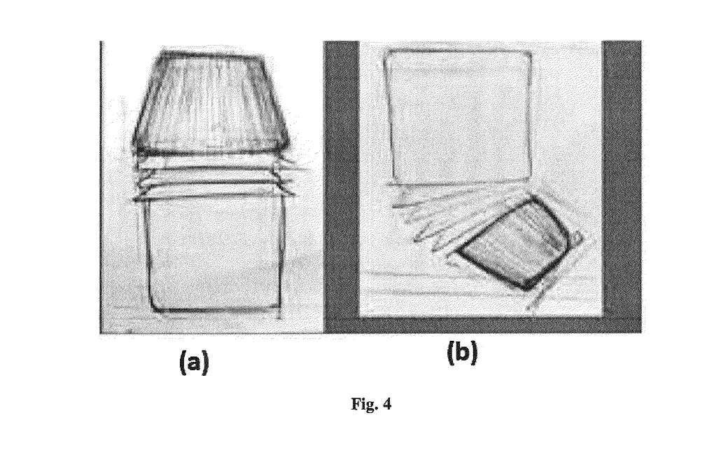

### DETAILED DESCRIPTION OF THE INVENTION

The examples shown here are intended only to illustrate some of the countless features of the invention and should not, however, limit its scope.

### Container for Applying Powder in the Innermost Region of Footwear

Container Format

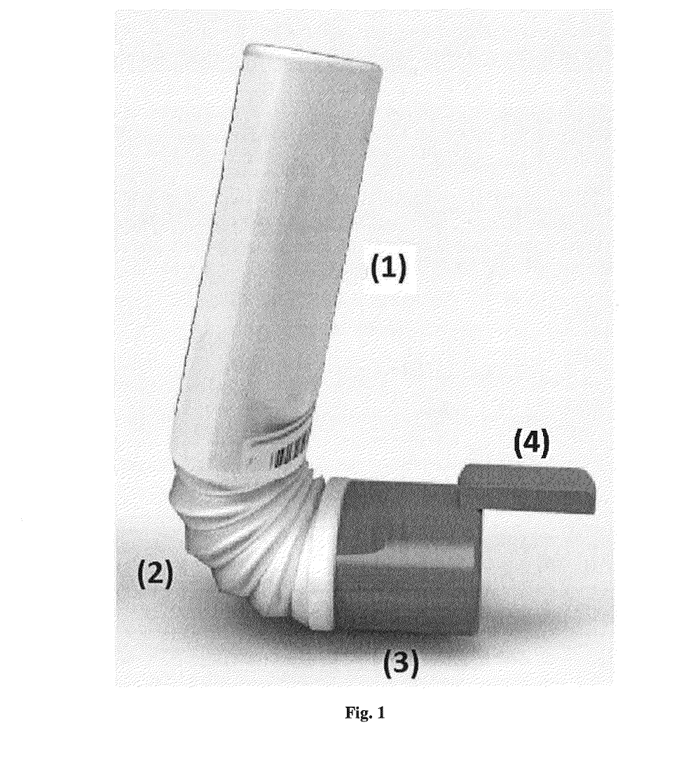

The container according to FIG. 1 can be divided into three distinct parts: the body (1), where the pressure to propel the powder composition is applied inside; the flexible and retractable neck (2); and the dispenser (3), comprising a cover (4).

The container material may be any known material in the prior art to enable the packaging and dispensing of the material. Preferred examples, but not limiting ones, include plastics.

Retractable Neck

The presence of longitudinal folds in the neck (2) of the vial allows the targeting of the dispenser (3) and in a different orientation of the container, and performs communication of these two spaces, so as to allow the free flow of the composition of the innermost region of the vial to the outside—difficult access regions in footwear. The neck (2) can still present an internal “graining”, to minimize dust loss for accommodation in the longitudinal folds.

Dispenser

The dispenser (3) allows the distribution of the composition within the container to the external environment, comprising a cover (4). This cover (4) may contain a single or a plurality of openings, providing a differentiated distribution when desired.

The composition on the inside of the container includes, with no limitation, any composition in the powder known by the prior art, useful for application within the footwear. In a preferred embodiment, the composition comprises triclosan and the undecylenic acid.

### Method for Applying Fluids

The method for applying fluids of the present invention comprises the steps of:

a) placing the device in the location where the fluid is applied by adjusting the retractable flexible neck; and

b) applying the fluid to the desired location.

The positioning of the device is preferably such that the dispensing orifice is oriented to the location of difficult access, for example, the inner region of a shoe.

As the user exerts pressure on the container, the fluid is expelled and, outwards, reaches the desired difficult access location.

### EXAMPLE 1

### Application of Deodorant Foot Powder

The container of this invention is used for applying deodorant foot powder. Thus, this powder has in its content undecylenic acid, zinc undecylenate and triclosan in combination.

The plastic container is prepared for example, polyethylene is prepared and it is constituted of a flexible, retractable neck for applying the talcum powder on the inside of long shoes.

In this example, the container is suitable for containing approximately 45 g of powder. Thus, the actives described above, in addition to the excipients, may total 45 g.

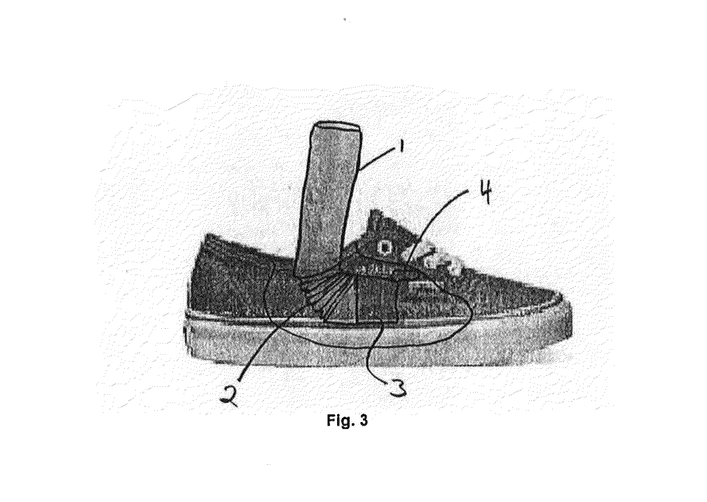

Such a formulation may be useful, as showed in FIG. 3, for the treatment of athlete's foot, bad smell and other problems related to the proliferation of microorganisms in the feet by prolonged suppression of the region.

Table 1 shows the components used in the example:

TABLE 1

| Substance | Classification |
|---|---|
| Polyethylene | Plastic (vial) |
| Undecylenic acid | Antifungal |
| Zinc Undecylenate | Antifungal |
| Triclosan | Antibiotic and Antifungal |

Those skilled in the art will value the knowledge presented herein and can reproduce the invention in the embodiments presented and in other variants, encompassed within the scope of the appended claims.

1. A device for applying fluid in difficult access locations, characterized in that it comprises a retractable, flexible region to facilitate the dispensing of fluid and can be oriented in up to 360°, remaining in the idle state when still.

2. The device according to claim 1, characterized in that the retractable and flexible region has longitudinal folds.

3. The device according to claim 1, characterized in that said fluid is a powder-based composition.

4. The device according to claim 3, characterized in that the powder composition comprises undecylenic acid, zinc undecylenate and triclosan.

5. The device according to claim 1, characterized in that it is produced by using recycled polyethylene.

6. The device according to claim 1, characterized in that it is used to treat foot odor.

7. The device according to claim 1, characterized in that it is used to treat athlete's foot.

8. A method for applying fluids in difficult access locations, characterized in that it comprises the steps of:
a) placing the device of claim 1 in the location where the fluid is applied by adjusting the retractable flexible neck; and
b) applying the fluid to the desired location.

9. The method according to claim 8, characterized in that the fluid is applied from an external force applied to the container which contains.
